(19) United States
(12) Patent Application Publication
(10) Pub. No.: US 2025/0239165 A1
(43) Pub. Date: Jul. 24, 2025

(54) Stress Mitigation Techniques Related to Physiological Measurements of Passengers During Travel

(71) Applicant: Textron Aviation Inc., Wichita, KS (US)

(72) Inventors: Rebecka Marie Mann, Wichita, KS (US); Lane Kirstein, Wichita, KS (US)

(21) Appl. No.: 18/420,893

(22) Filed: Jan. 24, 2024

(51) Int. Cl. G08G 5/00; A61B 5/16

(52) U.S. Cl. CPC G08G 5/34; A61B 5/165

(57) ABSTRACT

This disclosure provides methods, components, devices, and systems for mitigating anxiety of a passenger along a travel route while in a vehicle. Some aspects, more specifically, relate to detecting an anxiety level of a passenger during travel and implementing anxiety mitigating techniques to alleviate the anxiety. Travel anxiety can increase the stress of a passenger during travel, especially during air travel. In some aspects, a vehicle can analyze sensor data to detect a stress level of a passenger and determine a remedial action based on the stress level. Upon determining the remedial action, the vehicle can implement the remedial action so as to mitigate or alleviate the stress associated with the passenger while traveling in the vehicle.

### BACKGROUND

### 1. Field

This disclosure relates generally to stress mitigation techniques and, more specifically, to utilizing biotelemetry technology to obtain physiological measurements of a passenger and to implement stress mitigation techniques based on the physiological measurements.

### 2. Description of the Related Art

Many different types of passenger management systems and methods have been described in the prior art. For example, U.S. Pat. No. 11,479,147 to Jales Costa et al. describes systems and methods for occupant management for a vehicle, which includes an estimation module for determining passenger characteristics. U.S. Patent Application Publication No. 2018/01182189 to Miloser et al. and U.S. Patent Application Publication No. 2020/0239002 to Sobhany et al. both describe systems and methods for adapting vehicle operation based on passenger state. U.S. Pat. No. 11,587,357 to Kaliouby et al. describes systems and methods for collecting passenger cognitive data in a vehicle. U.S. Patent Application Publication No. 2007/0021885 to Soehren et al. describes systems and methods for personalizing the ride and handling characteristics of a vehicle.

### SUMMARY

Introduced here are methods, components, devices, and systems for stress mitigation techniques implementable on a vehicle for passengers experiencing stress-related symptoms.

One innovative aspect of the subject matter described in this disclosure can be implemented in a method for implementing stress mitigation techniques for passengers within a vehicle during travel. The method includes receiving sensor data associated with physiological measurements of a passenger within the vehicle. The method also includes determining a state of the passenger based, at least in part, on the physiological measurements of the passenger and calculating a remedial action associated with the state of the passenger and vehicle. The method further includes implementing the remedial action on the vehicle to alter the state of the passenger.

In some examples, where calculating the remedial action, the method includes inputting the sensor data and information associated with the vehicle and a travel route into a machine learning model trained on historical data associated with the vehicle and the passenger. The machine learning model can produce a remedial action inference. The method also includes utilizing the remedial action inference as the remedial action.

Another innovative aspect of the subject matter described in this disclosure can be implemented on a vehicle. The vehicle has a processing system that includes one or more processors and one or more memories coupled with the one or more processors. The processing system is configured to cause the vehicle to determine a state of a passenger based, at least in part, on sensor data associated with the physiological measurements of the passenger in the vehicle. The processing system is also configured to cause the vehicle to calculate a remedial action associated with the state of the passenger and the vehicle and implement the remedial action on the vehicle to alter the state of the passenger.

An additional innovative aspect of the subject matter described in this disclosure can be implemented in one or more computer storage media. The one or more computer storage media storing computer-useable instructions that, when executed by one or more computing devices, cause the one or more computing devices to perform operations, including receiving information associated with an aircraft and a passenger within the aircraft predicting a source of passenger stress associated with the information and based, at least in part, to the passenger and the aircraft. The one or more computer storage media storing computer-useable instructions also include calculating a preemptive remedial action associated with the source of passenger stress and implementing the preemptive remedial action on the aircraft.

This summary is intended to introduce a selection of concepts in a simplified form that is further described in the detailed description section of this disclosure. The summary is not intended to identify key or essential features of the claimed subject matter, nor is it intended to aid in determining the scope of the claimed subject matter. Additional objects, advantages, and novel features of the technology will be set forth in the following description and, in part, will become apparent to those skilled in the art upon examination of the disclosure or learned through practice of the technology.

### BRIEF DESCRIPTION OF THE DRAWINGS

These and other features, aspects, and advantages of the embodiments of the disclosure will become better understood with regard to the following description, appended claims, and accompanying drawings where:

FIG. 1 is a block diagram of an example stress mitigation system for providing remedial actions associated with stressful states of passengers during travel, in accordance with embodiments of the present disclosure.

FIG. 2 is an example avionics system, in accordance with embodiments of present disclosure.

FIG. 3 is an example flowchart for providing remedial actions associated with stressful states of passengers during travel, in accordance with embodiments of the present disclosure.

FIG. 4 is an example flowchart for providing remedial actions associated with potential sources of stress during travel, in accordance with embodiments of the present disclosure.

FIG. 5 is a schematic diagram of an example computing environment in which aspects of the present disclosure are employed in accordance with embodiments of the present disclosure.

FIG. 6 is a block diagram of an analysis computing entity of FIG. 4, in accordance with embodiments of the present disclosure.

FIG. 7 is a block diagram of a source computing entity(s) of FIG. 4, in accordance with embodiments of the present disclosure.

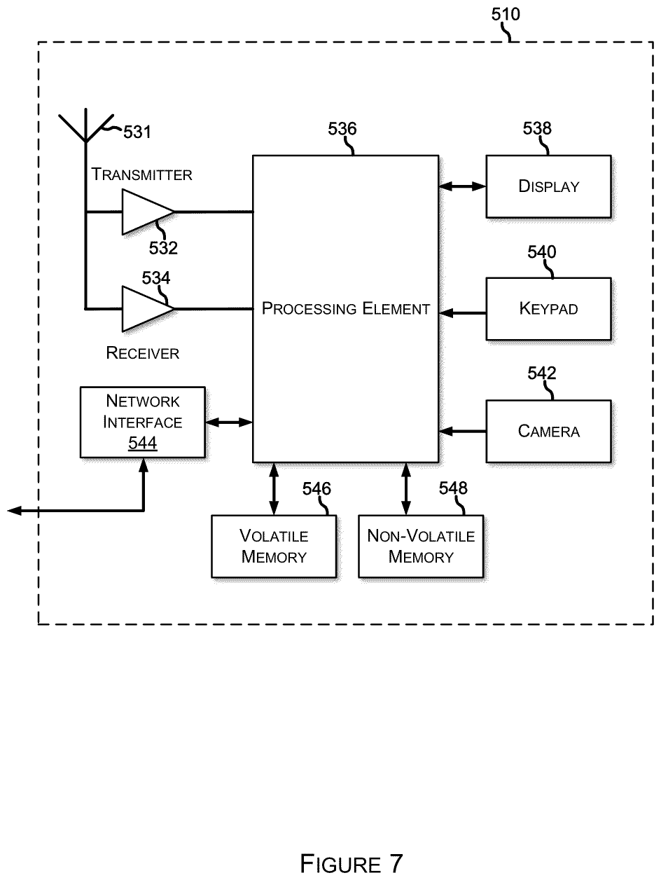

While the present disclosure is amenable to various modifications and alternative forms, specifics thereof, have been shown by way of example in the drawings and will be described in detail. It should be understood, however, that the intention is not to limit the particular embodiments described. On the contrary, the intention is to cover all modifications, equivalents, and alternatives falling within the scope of the present disclosure. Like reference numerals are used to designate like parts in the accompanying drawings.

### DETAILED DESCRIPTION

This disclosure relates generally to stress mitigation techniques and, more specifically, to utilizing biotelemetry sensor data to obtain physiological measurements of a passenger and to implement stress mitigation techniques based on the physiological measurements. The following description is directed to some particular examples for the purpose of describing innovative aspects of this disclosure. However, a person having ordinary skill in the art will readily recognize that the teachings herein can be applied in a multitude of different ways.

### Overview

Electric Vertical Takeoff and Landing (eVTOL) aircraft are a rapidly evolving technology, and several implementations involving passengers are being explored or are in development stages. These implementations are focused on transforming urban and regional transportation. For instance, one use of eVTOLs is as air taxis in urban environments. These services would provide quick, point-to-point transportation within cities, helping to alleviate road traffic congestion. In another example, e VTOLs could be used for rapid medical transport in urban and remote areas. Their ability to take off and land vertically makes them ideal for accessing difficult-to-reach locations, potentially improving response times in critical medical situations. In addition, many eVTOL aircraft are anticipated to be autonomous, lacking any human pilot or interface for control.

Passenger anxiety during flights, often referred to as flight anxiety or aviophobia, is a type of anxiety disorder that can occur in some individuals when they fly or anticipate flying. This condition can vary in intensity from mild nervousness to severe anxiety or panic attacks. The causes of flight anxiety can be varied and may include fear of heights, fear of enclosed spaces, past traumatic experiences related to flying, or lack of understanding about how flying works. It can also be related to broader anxiety disorders or stress-related issues.

Passenger anxiety can be triggered by various aspects of travel, such as flying, being in unfamiliar environments, or the anticipation of the journey itself. Passenger anxiety may be compounded by the autonomous nature of some eVTOL aircraft. The lack of a pilot in control could increase passenger concerns. The anxiety can manifest in several different ways. For instance, a person may experience physical symptoms, emotional symptoms, cognitive symptoms, and behavioral symptoms. Physical symptoms, as an example, can result in a person having an increased heart rate or palpitations, sweating, shaking or trembling, gastrointestinal distress, such as nausea, dizziness or lightheadedness, or shortness of breath or rapid breathing.

To manage passenger anxiety, individuals might use coping strategies like deep breathing, mindfulness meditation, or listening to calming music. Some might find it helpful to familiarize themselves with the travel process, plan thoroughly, and arrive early for departures. In more severe cases, counseling or therapy, such as cognitive-behavioral therapy (CBT) and, in some instances, medication, can be effective in managing travel-related anxiety.

Flight anxiety is a type of anxiety disorder that can occur in some individuals when they fly or anticipate flying. This condition can vary in intensity from mild nervousness to severe anxiety or panic attacks. The experience of flight anxiety can involve a combination of physical, emotional, and cognitive symptoms. A source of anxiety can be related to the takeoff and landing (TOL) portions of a flight.

Conventional remedies for flight anxiety, which range from psychological techniques to medical interventions, aim to help individuals manage their fear of flying and make air travel a more comfortable experience. Limitations on conventional remedies remain, however, as these remedies revolve around conditions that the passenger can modify or manipulate. For instance, the passenger may listen to music or attempt to sleep but cannot alter certain conditions specific to the vehicle or route. Speed, travel route, vehicle maneuvering, and altitude are some examples outside of the immediate control of the passenger. As such, challenges exist in providing robust stress mitigation techniques that effectively alleviate potential stressful conditions of passengers.

Various aspects of the disclosure improve existing technologies and techniques described herein, as well as others, by providing methods, components, systems, and devices that support the detection and remediation of stressful states of passengers during travel where the remediation is specific to the passenger's condition. Some aspects particularly relate to determining remedial actions based on the physiological measurements of passengers. During travel (e.g., a flight), passenger stress and anxiety can become elevated, sensor data from the vehicle or personal device can be collected and analyzed. Upon analysis of the physiological measurements, remedial actions can be calculated to assist in lowering the stress levels of a passenger. These remedial actions can be implemented directly on the vehicle during travel.

Particular aspects of the subject matter described in this disclosure can be implemented to realize one or more of the following potential advantages. The present disclosure aims to provide real-time stress mitigation solutions for passengers experiencing high-level stress associated with their travel experience by implementing mechanisms that ensure continuous monitoring and evaluation of passengers. By providing remedial actions implementable on a vehicle, the passengers can receive relief that would otherwise be unavailable. As an example, remedial actions implementable on an eVTOL aircraft transporting a passenger can result in the eVTOL aircraft restricting bank angles, restricting acceleration, restricting climb, restricting in-plane movement to reduce disorientation, rerouting the aircraft to avoid certain weather conditions, and slowing the transition from vertical to horizontal flight.

Certain aspects and techniques as described herein may be implemented, at least in part, using an artificial intelligence (AI) program, such as a program that includes a machine learning (ML) or Convolutional Neural Network (CNN) model. An example ML model may include mathematical representations or define computing capabilities for making inferences from input data based on patterns or relationships identified in the input data. As used herein, the term “inferences” can include one or more decisions, predictions, determinations, or values, which may represent outputs of the ML model. The computing capabilities may be defined in terms of certain parameters of the ML model, such as weights and biases. Weights may indicate relationships between certain input data and certain outputs of the ML model, and biases are offsets that may indicate a starting point for the outputs of the ML model. An example ML model operating on input data may start an initial output based on the biases and then update its output based on a combination of the input data and the weight.

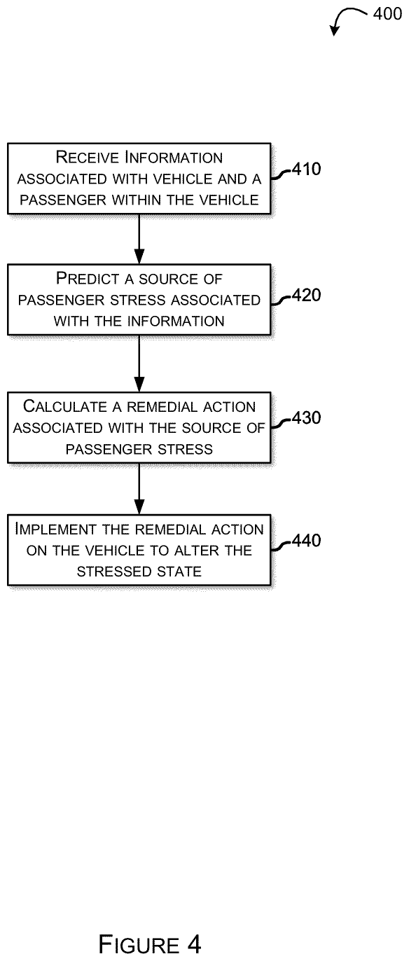

ML models may be deployed in one or more devices (for example, high-performance computers, servers, network infrastructure, and edge computing devices (e.g., an analysis computing entity of FIG. 4, a source computing entity of FIG. 4) and may be configured to enhance various aspects of a stress mitigation system. For example, an ML model may be trained to predict remedial actions in data corresponding to historical data and sensor data (e.g., a physiological sensor measuring temperature, heart rate, and movement). An ML model may support operational decisions relating to one or more aspects associated with detecting and resolving stressful states of passengers that can occur while traveling. For example, an ML model may be utilized for supporting or improving aspects such as remedial actions and stress state detection in passengers.

### Example Stress Mitigation System

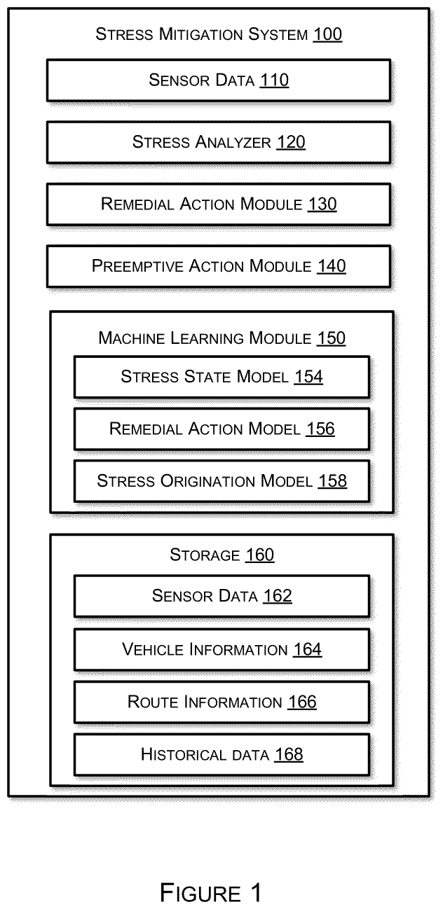

FIG. 1 shows a block diagram of an example stress mitigation system 100 suitable for use in implementing embodiments of the disclosure. The stress mitigation system 100 is configured to monitor passengers during travel and determine whether a passenger is in an elevated stressful state. The stress mitigation system 100 is further configured to calculate a remedial action to alleviate the stressful state of a passenger and to implement that remedial action on a vehicle. According to some aspects, the stress mitigation system 100 can include sensor data 110, a stress analyzer 120, a remedial action module 130, a preemptive action module 140, a machine learning module 150, and storage 160. The machine learning module can include a stress state model 154, a remedial action model 156, and a stress origination model 158. The storage can include sensor data 162, vehicle information 164, route information 166, and historical data 168.

The sensor data 110 is data or information from sensors associated with a passenger and/or vehicle. As an example, the sensor data 110 can be from a personal device, such as a smartphone, smartwatch, or fitness tracker, equipped with a variety of sensors that collect different types of data. These sensors include accelerometers, gyroscopes, magnetometers, ambient light sensors, barometers, temperature sensors, humidity sensors, proximity sensors, heart rate sensors, pedometers, GPS receivers, fingerprint sensors, facial recognition sensors, microphones, cameras, and the like.

In some examples, the sensor data 110 includes data from sensors positioned in and around a vehicle. These sensors include, but are not limited to, occupancy sensors, weight sensors, infrared sensors, capacitive sensors, cameras, image sensors, CO2 sensors, ultrasonic sensors, and health monitoring sensors. Health monitoring sensors, as an example, can include heart rate monitors, breath analyzers, blood pressure monitors, skin conductance sensors, oxygen saturation sensors, fatigue detection systems, temperature sensors, posture sensors, ECG sensors, and glucose monitoring.

In some aspects, the sensor data 110 can be received either directly from the sensors mentioned-above in configurations where the sensors are communicatively coupled to the stress mitigation system 100. In some aspects, the sensor data 110 can be received from another system used in the collection of sensor data.

The stress analyzer 120 is a component of the stress mitigation system 100 configured to analyze the sensor data (e.g., the physiological measurements) associated with a passenger and determine whether the passenger is in a stressful state. Stress typically causes an increase in heart rate. As an example, the stress analyzer 120 can monitor and evaluate various indicators, such as elevated heart rates, especially in the absence of physical exertion, which can be a strong indicator of stress. Another example includes Heart Rate Variability (HRV). HRV is the variation in the time interval between heartbeats. Lower HRV is often associated with higher stress levels, indicating a less adaptable cardiovascular system.

In some aspects, the stress analyzer 120 can also determine additional stress indicators detectable from sensor data, including skin conductance, also known as galvanic skin response (GSR), which measures the skin's electrical conductance, which increases with moisture (sweating). Stress can lead to increased sweating and, thus, higher skin conductance. Stress can cause an increase in blood pressure. Continuous or frequent monitoring of blood pressure can help in identifying stress-related changes. Stress often affects breathing patterns, leading to faster or shallower breathing.

Monitoring respiratory rates can, therefore, be a useful stress indicator. Stress responses can sometimes cause fluctuations in body temperature, typically an increase, which can be monitored via temperature sensors. Cortisol is a hormone released in response to stress. While not commonly monitored in standard health sensors due to the complexity of measurement, advanced systems or dedicated health devices might track cortisol levels. Stress can alter a person's voice, including changes in pitch, tone, and cadence. Voice analysis through microphone sensors can detect these changes.

The stress analyzer 120 can also utilize cameras equipped within the vehicle or personal device to analyze facial expressions and eye movements for signs of stress, such as frowning, blinking frequently, or having a tense expression. Wearable sensors can detect increased muscle tension, a common physical response to stress. Changes in regular patterns, like increased restlessness detected through motion sensors, or changes in daily routines, can also indicate stress.

In some aspects, the stress analyzer 120 can determine a stressful state of a passenger using a combination of sensor data. By integrating data from multiple sources, it can be possible to get a more accurate indication of a passenger's stress level. For example, combining heart rate data with HRV provides a more nuanced view of the cardiovascular response to stress. While an increased heart rate can indicate stress, incorporating HRV, which measures the time variation between heartbeats, can give insights into the body's autonomic nervous system regulation, a key component in stress response. In another example, skin conductance increases with sweating, a physiological response to stress, while stress can also lead to changes in peripheral body temperature. Monitoring skin conductance and temperature in tandem can offer corroborative evidence of stress.

Stress can cause both an increase in heart rate and changes in breathing patterns (such as more rapid or shallow breathing). Monitoring these together can help distinguish stress from physical exertion, which increases heart rate but typically involves deeper, more regular breathing.

In some aspects, the stress analyzer 120 can also determine additional stress indicators from a combination of sensor data including voice analysis and facial expression recognition. Changes in voice patterns (like pitch and tone) combined with facial expression analysis (such as tension in facial muscles or frowning) can provide strong behavioral indicators of stress, especially when analyzed in real-time. Another example includes muscle tension and movement patterns. Wearable sensors that detect muscle tension, combined with sensors that monitor overall movement and posture, allow the stress analyzer 120 to identify physical manifestations of stress, such as increased restlessness or muscle stiffness.

In some aspects, the stress analyzer 120 utilizes the machine learning module 150 using a stress state machine learning model 154 to determine the stressful state of a passenger. The machine learning module 150 is configured to preprocess the sensor data 110 (like normalization or feature extraction), select the right features and tune model parameters. Once the sensor data 110 is preprocessed, the machine learning module 150 can train the stress state model 154 to predict a stressful state of a passenger.

The stress state model 154 can be implemented as various machine learning models trained to analyze sensor data related to stress detection. The models include, but are not limited to, Support Vector Machines (SVM), random forests, neural networks, CNNs, Recurrent Neural Networks (RNN)s, Gradient Boosting Machines (GBM)s, and time-series analysis models. As an example, random forest learning models involve an ensemble learning method, which operates by constructing a multitude of decision trees and is suitable for handling large datasets with many input variables (like multiple sensor readings). Random forests are robust against overfitting and can provide insights into the importance of different sensors in predicting stressful states. Another example of the stress state model 154 is the RNN. These models are specifically designed for sequential data, making them ideal for analyzing time-series sensor data. Long Short-Term Memory (LSTM)s are a special kind of RNN capable of learning long-term dependencies, which can be particularly useful in detecting stress patterns over time.

The remedial action module 130 is a component of the stress mitigation system 100 configured to determine a remedial action associated with a stressful state of a passenger. Once the stress analyzer 120 determines the stressful state of a passenger, the remedial action module 130 can calculate a remedial action associated with the stressful state of the passenger. In some aspects, the remedial action module 130 factors in the level of stress of the passenger in calculating the remedial action. For instance, if a passenger has a slightly elevated stressful level, then the remedial action can include making minor adjustments to the internal environment of the vehicle. This can include adjusting the temperature, modifying the ambient lighting, playing soothing music, increasing the oxygen level, and the like.

In some aspects, the remedial action module 130 factors in the type of vehicle transporting the passenger. For instance, the vehicle may be an eVTOL aircraft. Passengers, in this instance, may have an initial heightened level of stress due to the autonomous nature of the aircraft. As such, the remedial action module 130 can calculate a remedial action that results in the aircraft modifying its flight envelope. The flight envelope of an aircraft can refer to the range of its capabilities in terms of airspeed, altitude, load, and maneuverability. It can be a graphical representation that shows the limits within which an aircraft can safely operate.

Some examples of a remedial action altering a flight envelope include restricting bank angles of an aircraft to shallower banks, restricting the max acceleration or deceleration of the aircraft, restricting the max climb or descend rate, restricting in-plane movement to reduce disorientation, rerouting the aircraft to avoid weather conditions or weather patterns (e.g., thunderstorms, known turbulence), and the like.

As an example, eVTOL aircraft are unique compared to traditional fixed wing, rotorcraft, or ground-based vehicles in that they have an extremely high degree of simultaneous maneuverability in the X-Y-Z axes. Remedial actions altering a flight envelope can be extended to restriction combination maneuvers that are not possible in traditional vehicles. For example, a typical landing for an eVTOL vehicle may involve a transition from horizontal to vertical flight while simultaneously descending and maneuvering in-plane. As such, remedial actions altering the flight envelope may involve performing each maneuver as a part of an eVTOL landing separately instead of simultaneously. In order to fulfill their role in urban commuting it is expected that eVTOL aircraft may be allowed to operate in a wider range of airspace than traditional flight vehicles. eVTOL aircraft may be expected to fly much closer to buildings in an urban center and operate in higher population density areas. Additional examples of remedial actions that alter the flight envelope may also involve allowing more spacing between the e VTOL and buildings or high population areas to reduce passenger stress associated with flight in close proximity to obstacles.

In some aspects, the remedial action module 130 factors historical data 168 when calculating the remedial action. Historical data can include previous physiological measurements of the passenger, previous stressful states of a passengers, previous remedial actions taken, prior travel conditions of the route, and information relating to specific passengers, their prior stress states, and remedial actions taken. Based on the historical data, the remedial action module 130 can calculate a remedial action that can be catered to a specific passenger, travel route, and vehicle.

In some aspects, the remedial action module 130 utilizes the machine learning module 150 using a remedial action model 156 to calculate a remedial action. Similar to the stress state model 154, the machine learning module 150 is configured to preprocess the sensor data 110 (like normalization or feature extraction), select the right features and tune model parameters. Once the sensor data 110 is preprocessed, the machine learning module 150 can train the remedial action model 156 to predict a remedial action. The machine learning module 150 can also preprocess additional information such as vehicle information 164, route information 166, and historical data 168 that can also be used by the remedial action model 156 in predicting a remedial action.

The remedial action model 156 can be implemented as various machine learning models trained to calculate remedial actions related to stressful states of passengers. These machine learning models include, but are not limited to, deep learning models (e.g., RNNs, CNNs), random forests, GBMs, recommendation systems, reinforcement learning models, and multi-modal fusion models.

The models mentioned above can be trained by the machine learning module 150 to accurately assess stress levels and understand the context and possible remedial actions. In some aspects, the machine learning module 150 can implement the remedial action model 156 as a hybrid approach that combines several machine learning techniques. In some aspects, the machine learning module 150 provides the remedial action model 156 with data associated with continuous monitoring of the passenger and updates the remedial action model 156 with new data and feedback to adapt to changing scenarios and individual needs.

The preemptive action module 140 is a component of the stress mitigation system 100 configured to determine remedial actions that can be preemptively implemented during travel to prevent in an increase in stress of a passenger. Taking factors into consideration, such as the route, the vehicle, the passenger, and travel conditions, the preemptive action module 140 can calculate a remedial action. In some aspects, the preemptive action module 140 utilizes historical data relating to the factors mentioned above as well as historical data produced by the stress analyzer 120 and the remedial action module 130 in calculating the remedial action. For instance, if the stress analyzer 120 has analyzed a passenger previously and the remedial action module 130 calculated a remedial action, then that historical data can be used by the preemptive action module 140 in calculating its preemptive remedial action.

In some aspects, the preemptive remedial action calculated by the preemptive action module 140 is similar to the remedial actions of the remedial action module 130. For instance, if passengers have historically had a slightly elevated stressful level during a particular route or vehicle, then the remedial action can include making minor adjustments to the internal environment of the vehicle. This can include adjusting the temperature, modifying the ambient lighting, playing soothing music, increasing the oxygen level, and the like.

In some aspects, the preemptive action module 130 factors in the type of vehicle transporting the passenger. For instance, the vehicle may be an eVTOL aircraft. Passengers, in this instance, may historically have an initial heightened level of stress due to the autonomous nature of the aircraft. As such, the preemptive action module 130 can calculate a remedial action that results in the aircraft modifying its flight envelope that has historically prevented or alleviated stress in passengers.

As mentioned, in some aspects, the preemptive action module 130 factors historical data 168 when calculating the preemptive remedial action. Historical data can include previous physiological measurements of the passenger, previous stressful states of a passengers, previous remedial actions taken, prior travel conditions of the route, and information relating to specific passengers, their prior stress states, and remedial actions taken. Based on the historical data, the preemptive action module 130 can calculate a preemptive remedial action that can be catered to a specific passenger, travel route, and vehicle that can be implemented to avoid passenger stress.

In some aspects, the preemptive action module 130 utilizes the machine learning module 150 to utilize a stress origination model 158 to calculate a source of passenger stress and recommend a preemptive remedial action. Similar to the stress state model 154 and the remedial action model 156, the machine learning module 150 is configured to preprocess information such as vehicle information 164, route information 166, and historical data 168 (including historical passenger stress, causes of passenger stress, and associated remedial actions). Once the information is preprocessed, the machine learning module 150 can train the stress origination model 158 to predict sources of passenger stress based on factors such as passenger history, vehicle, route, and weather conditions.

The stress origination model 158 can be implemented as various machine learning models trained to calculate remedial actions related to stressful states of passengers. These machine learning models include, but are not limited to, deep learning models (e.g., RNNs, CNNs), random forests, GBMs, recommendation systems, reinforcement learning models, and multi-modal fusion models.

It is noted that FIG. 1 is intended to depict the major representative components of a stress mitigation system 100. In some embodiments, however, individual components may have greater or lesser complexity than, as represented in FIG. 1, components other than or in addition to those shown in FIG. 1 may be present, and the number, type, and configuration of such components may vary.

### Example Avionics System

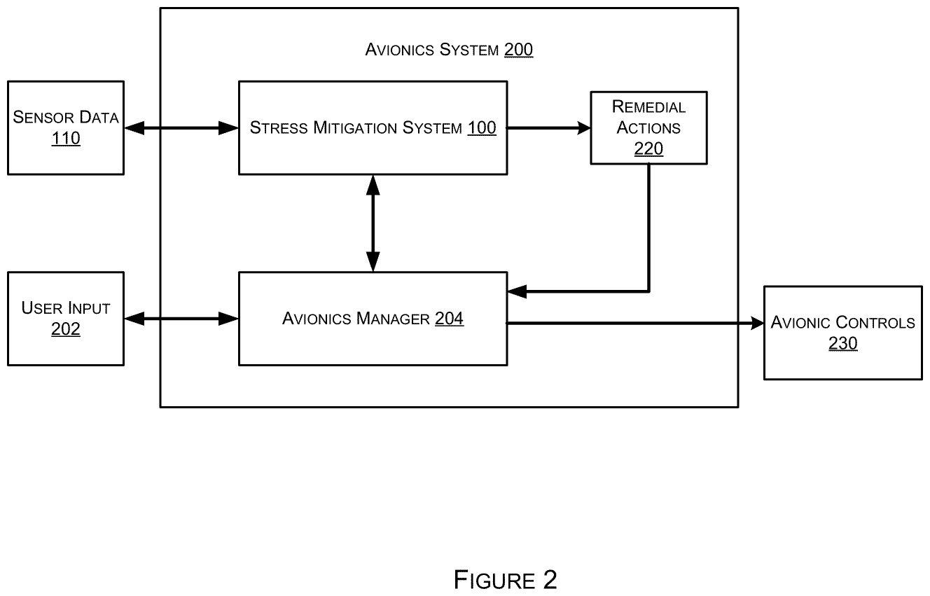

FIG. 2 illustrates an example avionics system 200 using a stress mitigation system 100, in accordance with embodiments of the present disclosure. As discussed, the techniques described herein provide remedial actions associated with detecting and determining the stressful states of passengers during travel. Accordingly, in some embodiments, a vehicle or personal device can provide the sensor data 110 (i.e., the physiological measurements) to the stress mitigation system 100 to determine whether a passenger is in a stressful state. If detected, the stress mitigation system 100 can calculate a remedial action 220 associated with the stressful state.

As shown in FIG. 2, optionally, the stress mitigation system 100 is implemented as part of an avionics system 200. Alternatively, the stress mitigation system 100 is implemented in a separate system that provides at least the remedial actions 220 (e.g., recommendations, preemptive remedial action) to the avionics system 200 for implementation. The avionics system 200 is configured to encompass a wide range of electronic systems used for communication, navigation, monitoring and managing multiple systems, and providing various flight-related services.

The electronic systems (not shown) include, but are not limited to, GPS systems, Inertial Navigation Systems (INS), VHF Omnidirectional Range (VOR) and Instrument Landing Systems (ILS), communication systems (e.g., radios, satellite communications), flight-control systems, flight management systems, and weather systems.

Once the output 220, representing the remedial actions for stressful states of passengers, has been obtained by the avionic system 200, the output 220 can be either directly implemented or reviewed and evaluated by a pilot or authorized personnel. In some aspects, user input 202 (e.g., travel preferences) can be received by an avionics manager 204 of the avionics system 200. The user input 202 can include any information regarding a travel route, a vehicle, particularities of a passenger, and passenger preferences that can be used by the stress mitigation system 100 when producing the remedial actions 220. The avionics manager 204 can also generate avionic controls 230 known in the art and may include the remedial actions 220 produced by the stress mitigation system 100 as a result of evaluating the sensor data 110 and the user input 202.

In some aspects, the resulting remedial actions 220 generated by the stress mitigation system 100 may be output to a different or downstream avionics system for evaluation and implementation. The implementation may take any discussed remedial actions possible on the vehicle or aircraft, including output to authorized personnel on the vehicle, such as via a user interface when the remedial action 220 indicates a change to a flight envelope. The user interface may include user interface elements for drilling down into the details of the remedial action, including identifying the various recommendations and stressful state of a passenger. In this way, the authorized personnel may identify which features of the remedial action 220 to implement and whether the stressful state of the passenger requires medical attention. User interface elements may allow authorized personnel to provide input to indicate approval for implementing the remedial actions 220.

### Example Flow Diagram

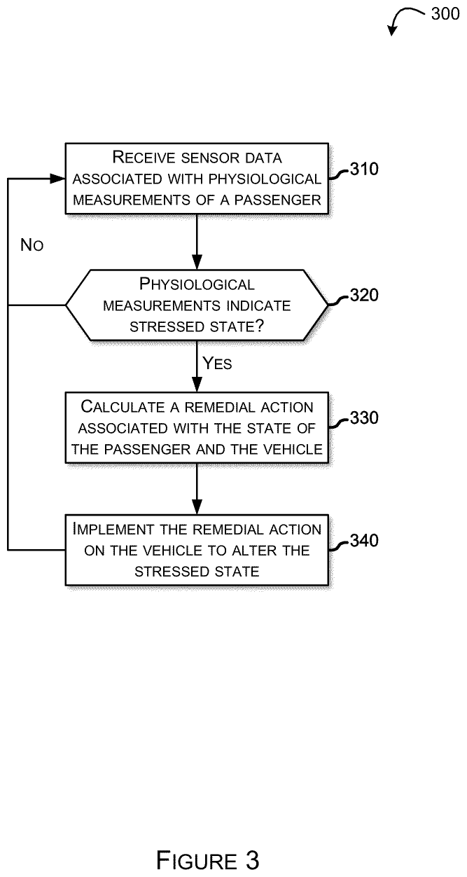

With reference to FIG. 3, a flow diagram illustrating a method is provided. Each block of the method 300 and any other methods described herein include a computing process performed using any combination of hardware, firmware, and/or software. For instance, in some embodiments, various functions are carried out by a processor executing instructions stored in memory. In some cases, the methods are embodied as computer-usable instructions stored on computer storage media.

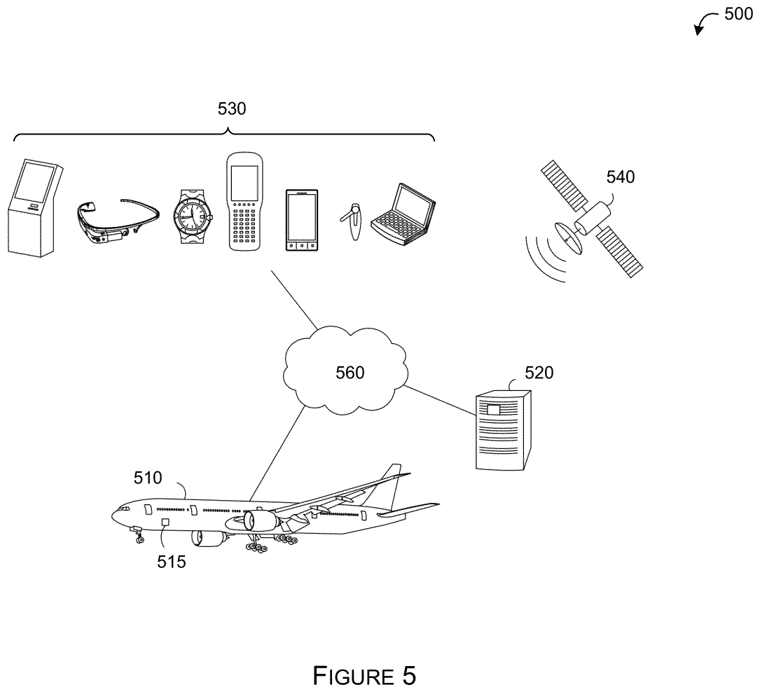

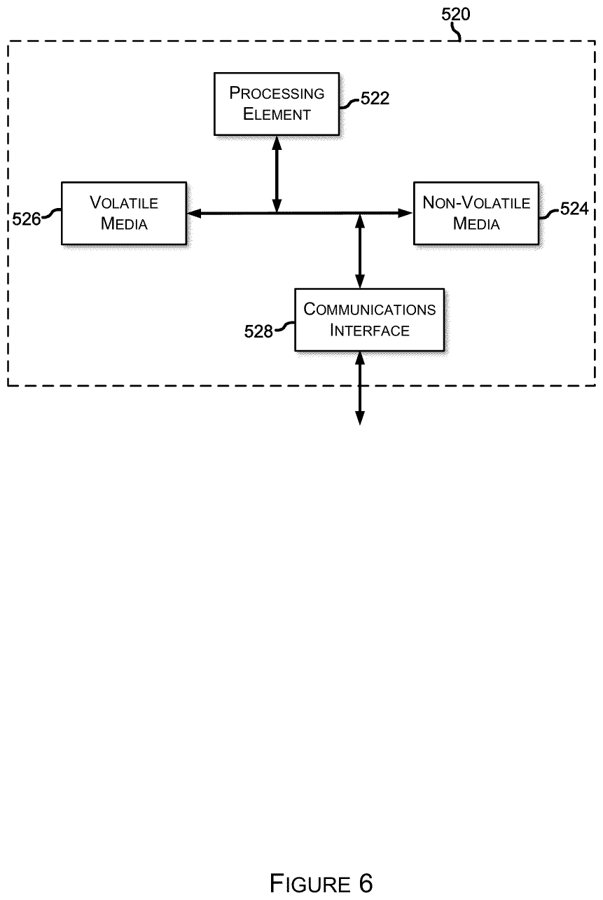

FIG. 3 illustrates a method 300 of a series of acts in a method of implementing a remedial action on a vehicle based on a stressful state of a passenger, in accordance with embodiments of the present disclosure. In one or more embodiments, the method 300 is performed in a digital medium environment that includes the stress mitigation system 100. In some examples, the method 300 may be performed by a computing device such as the one of the computing devices described with reference to FIGS. 5 and 6. The method 300 is intended to be illustrative of one or more methods in accordance with the present disclosure and is not intended to limit potential embodiments. Alternative embodiments can include additional, fewer, or different steps than those articulated in FIG. 3.

As illustrated in FIG. 3, the method 300 includes a block 310 that receives sensor data associated with physiological measurements of a passenger. As described, the sensor data can include data from accelerometers, gyroscopes, magnetometer, ambient light sensors, barometers, temperature sensors, humidity sensors, proximity sensors, heart rate sensors, pedometers, GPS receivers, fingerprint sensors, facial recognition sensors, microphones, cameras, and the like.

In some aspects, the sensor data can also include data from occupancy sensors, weight sensors, infrared sensors, capacitive sensors, cameras, image sensors, CO2 sensors, ultrasonic sensors, and health monitoring sensors.

At block 320, the stress analyzer 120 of FIG. 1 determines whether a passenger in a vehicle is in a stressful state. In some aspects, the stress analyzer 120 analyzes the physiological measurements associated with the passenger to determine whether or not the physiological measurements indicate a stressful state. If no stress is detected, the stress mitigation system 100 of FIG. 1 can continuously monitor the sensor data to detect stress in the passenger. However, if the stress analyzer 120 detects stress, the method 300 proceeds to block 330.

At block 330, the remedial action module 130 calculates a remedial action associated with the stressful state of the passenger. As discussed, and in some aspects, the remedial action module 130 can factor in the sensor data, the stressful state of the passenger, the vehicle, the route, and historical data to calculate an optimized remedial action that can alleviate the stressful state of the passenger. At block 340, the stress mitigation system 100 implements the remedial action on the vehicle. In some aspects, the stress mitigation system 100 transmits the remedial action to an avionics system of an aircraft. The remedial action can include recommendations that involve altering the internal environment of the vehicle or aircraft (e.g., lighting, temperature, music) as well as recommendations for altering the flight envelope of the vehicle. As discussed, this can include restricting bank angles of an aircraft to shallower banks, restricting the max acceleration or deceleration of the aircraft, restricting the max climb or descend rate, restricting in-plane movement to reduce disorientation, rerouting the aircraft to avoid weather conditions or weather patterns (e.g., thunderstorms, known turbulence), and the like.

With reference to FIG. 4, a flow diagram illustrating a method is provided. Each block of the method 400 and any other methods described herein include a computing process performed using any combination of hardware, firmware, and/or software. For instance, in some embodiments, various functions are carried out by a processor executing instructions stored in memory. In some cases, the methods are embodied as computer-usable instructions stored on computer storage media.

FIG. 4 illustrates a method 400 of a series of acts in a method of implementing a preemptive remedial action on a vehicle based on information associated with a passenger and a vehicle (e.g., prior and historical information), in accordance with embodiments of the present disclosure. In one or more embodiments, the method 400 is performed in a digital medium environment that includes the stress mitigation system 100. In some examples, the method 400 may be performed by a computing device such as the one of the computing devices described with reference to FIGS. 5 and 6. The method 400 is intended to be illustrative of one or more methods in accordance with the present disclosure and is not intended to limit potential embodiments. Alternative embodiments can include additional, fewer, or different steps than those articulated in FIG. 5.

As illustrated in FIG. 4, the method 400 includes a block 410 that receives information associated with a vehicle (e.g., an aircraft) and a passenger within the vehicle. This information can include, but is not limited to, historical sensor data of passengers, including the passenger, vehicle data, route information, historical remedial actions, historical remedial actions taken within the vehicle or similar vehicles, historical remedial actions taken along the travel route, historical stressful states of passengers including the passenger, and the like.

At block 420 the preemptive action module 140 of FIG. 1 predicts a source of passenger stress associated with the information. For instance, the information may indicate that passengers typically become stressed along a certain portion of the route, or when the vehicle performs certain maneuvers, or that the passenger typically becomes stressed during travel. In some aspects, the preemptive action module 140 utilizes a machine learning model to perform predictions. The machine learning model can be trained using historical information to make predictions of sources of passenger stress.

At block 430, the preemptive action module 140 calculates a preemptive remedial action associated with the source of passenger stress. As discussed, and in some aspects, the preemptive action module 140 can factor in historical information such as historical sensor data, historical stressful states of the passenger, the vehicle, the route, and other additional historical data to calculate an optimized preemptive remedial action that can prevent a stressful state of the passenger. At block 440, the stress mitigation system 100 implements the preemptive remedial action on the vehicle. In some aspects, the stress mitigation system 100 transmits the preemptive remedial action to an avionics system of an aircraft. The preemptive remedial action can include recommendations that involve altering the internal environment of the vehicle or aircraft (e.g., lighting, temperature, music) as well as recommendations for altering the flight envelope of the vehicle.

The invention may be described in the general context of computer code or machine-useable instructions, including computer-executable instructions such as program modules, being executed by a computer (or one or more processors) or other machine, such as a personal data assistant or other handheld device. Generally, program modules including routines, programs, objects, components, data structures, etc., refer to code that perform particular tasks or implement particular abstract data types. The invention may be practiced in a variety of system configurations, including hand-held devices, consumer electronics, general-purpose computers, more specialty computing devices, etc. The invention may also be practiced in distributed computing environments where tasks are performed by remote-processing devices that are linked through a communications network.

### Computer Program Products, Methods, and Computing Entities

Embodiments of the present disclosure may be implemented in various ways, including as computer program products that comprise articles of manufacture. A computer program product may include a non-transitory computer-readable storage medium storing applications, programs, program modules, scripts, source code, program code, object code, byte code, compiled code, interpreted code, machine code, executable instructions, and/or the like (also referred to herein as executable instructions, instructions for execution, program code, and/or similar terms used herein interchangeably). Such non-transitory computer-readable storage media include all computer-readable media (including volatile and non-volatile media).

In one embodiment, a non-volatile computer-readable storage medium may include a floppy disk, flexible disk, hard disk, solid-state storage (SSS) (e.g., a solid-state drive (SSD), solid state card (SSC), solid state module (SSM)), enterprise flash drive, magnetic tape, or any other non-transitory magnetic medium, and/or the like. A non-volatile computer-readable storage medium may also include a punch card, paper tape, optical mark sheet (or any other physical medium with patterns of holes or other optically recognizable indicia), compact disc read only memory (CD-ROM), compact disc-rewritable (CD-RW), digital versatile disc (DVD), Blu-ray disc (BD), any other non-transitory optical medium, and/or the like. Such a non-volatile computer-readable storage medium may also include read-only memory (ROM), programmable read-only memory (PROM), erasable programmable read-only memory (EPROM), electrically erasable programmable read-only memory (EEPROM), flash memory (e.g., Serial, NAND, NOR, and/or the like), multimedia memory cards (MMC), secure digital (SD) memory cards, SmartMedia cards, CompactFlash (CF) cards, Memory Sticks, and/or the like. Further, a non-volatile computer-readable storage medium may also include conductive-bridging random access memory (CBRAM), phase-change random access memory (PRAM), ferroelectric random-access memory (FeRAM), non-volatile random-access memory (NVRAM), magnetoresistive random-access memory (MRAM), resistive random-access memory (RRAM), Silicon-Oxide-Nitride-Oxide-Silicon memory (SONOS), floating junction gate random access memory (FJG RAM), Millipede memory, racetrack memory, and/or the like.

In one embodiment, a volatile computer-readable storage medium may include random access memory (RAM), dynamic random access memory (DRAM), static random access memory (SRAM), fast page mode dynamic random access memory (FPM DRAM), extended data-out dynamic random access memory (EDO DRAM), synchronous dynamic random access memory (SDRAM), double information/data rate synchronous dynamic random access memory (DDR SDRAM), double information/data rate type two synchronous dynamic random access memory (DDR2 SDRAM), double information/data rate type three synchronous dynamic random access memory (DDR3 SDRAM), Rambus dynamic random access memory (RDRAM), Twin Transistor RAM (TTRAM), Thyristor RAM (T-RAM), Zero-capacitor (Z-RAM), Rambus in-line memory module (RIMM), dual in-line memory module (DIMM), single in-line memory module (SIMM), video random access memory (VRAM), cache memory (including various levels), flash memory, register memory, and/or the like. It will be appreciated that where embodiments are described to use a computer-readable storage medium, other types of computer-readable storage media may be substituted for or used in addition to the computer-readable storage media described above.

As should be appreciated, various embodiments of the present disclosure may also be implemented as methods, apparatus, systems, computing devices/entities, computing entities, and/or the like. As such, embodiments of the present disclosure may take the form of an apparatus, system, computing device, computing entity, and/or the like executing instructions stored on a computer-readable storage medium to perform certain steps or operations. However, embodiments of the present disclosure may also take the form of an entirely hardware embodiment performing certain steps or operations.

Embodiments of the present disclosure are described below with reference to block diagrams and flowchart illustrations. Thus, it should be understood that each block of the block diagrams and flowchart illustrations may be implemented in the form of a computer program product, an entirely hardware embodiment, a combination of hardware and computer program products, and/or apparatus, systems, computing devices/entities, computing entities, and/or the like carrying out instructions, operations, steps, and similar words used interchangeably (e.g., the executable instructions, instructions for execution, program code, and/or the like) on a computer-readable storage medium for execution. For example, retrieval, loading, and execution of code may be performed sequentially such that one instruction is retrieved, loaded, and executed at a time. In some exemplary embodiments, retrieval, loading, and/or execution may be performed in parallel such that multiple instructions are retrieved, loaded, and/or executed together. Thus, such embodiments can produce specifically-configured machines performing the steps or operations specified in the block diagrams and flowchart illustrations. Accordingly, the block diagrams and flowchart illustrations support various combinations of embodiments for performing the specified instructions, operations, or steps.

### Example Computing Environment

FIG. 5 is a schematic diagram of an example computing environment 500 in which aspects of the present disclosure are employed, according to some embodiments. As shown in FIG. 5, this particular computing environment 500 includes one or more aircraft 510, one or more analysis computing entities 520, one or more source computing entities 530, one or more satellites 540, a communications network 560, and the like. Each of these components, entities, devices, systems, and similar words used herein interchangeably may be in direct or indirect communication with, for example, one another over the same or different wired or wireless networks. Additionally, while FIG. 5 illustrates the various system entities as separate, standalone entities, the various embodiments are not limited to this particular architecture.

The analysis computing entity(s) 520 is generally responsible for data monitoring and analysis of a vehicle (e.g., the aircraft 510) using a stress mitigation system and initializing the stress mitigation system by associating devices and data metrics relating to passengers within the vehicle. During runtime, the components are continuously updated with collected data metrics (e.g., sensor data, historical data) and the state of the passengers are analyzed by the systems to determine if the stress levels of a passenger require a mitigation technique be implemented on the vehicle.

During runtime, the components are continuously updated with collected data metrics and the state of the passengers are analyzed by the systems to determine if an event has occurred corresponding requiring a remedial action. The remedial actions can correspond to actions occurring on the vehicle as they relate to the passengers associated with the determined states. Remedial actions can also correspond to steps that are taken to rectify the state of a passenger.

The source computing entity(s) 530 (or user device) is generally responsible for providing physiological measurements using device sensors positioned within the source computing entity(s) 530. For example, in response to the source computing entity(s) 530 measuring a physiological measurement of a passenger, the source computing entity(s) 530 can provide, over the network(s) 560, to either the analysis computing entity(s) 520, or the aircraft 510, the physiological measurements associated with a passenger. It is understood that in some embodiments, the source computing entity(s) 530 alternatively or additionally provide the physiological measurements to the aircraft 510 and/or utilizes services offered by the stress mitigation system 100.

In various embodiments, the aircraft 510 includes one or more telematics sensor devices 515. The telematics sensor devices 515 sample various attributes of the aircraft 510, such as speed, location (e.g., via a GPS module), fuel consumption, and/or any other suitable type attributes of the vehicle 510 and transmit the data to the analysis computing entity 520 for analysis. In some embodiments, any of the historical flight data described herein, such as FPS data, is automatically derived via the telematics sensor devices 515. For example, in order to determine where a particular aircraft flew, how many miles were traversed, etc. a GPS or other module that acts as a telematics sensor device 520 may sample such data and store, in near-real-time, such data as a stream of values.

In various embodiments, the aircraft 510 includes one or more physiological sensor devices (not shown). In some embodiments, the physiological sensor devices can continuously measure a passenger's heart rate, blood pressure, respiratory rate, oxygen saturation, ECG/EKG, EEG, EMG, body temperature, blood glucose levels, capnography, pulse rate, and/or any other suitable type of physiological measurement and transmit the data to the analysis computing entity 520 for analysis.

### Example Analysis Computing Entity

FIG. 5 is a block diagram of the analysis computing entity 520 of FIG. 5, according to particular embodiments of the present disclosure. In general, the terms computing entity, computer, entity, device, system, and/or similar words used herein interchangeably may refer to, for example, one or more computers, computing entities, desktops, mobile phones, tablets, phablets, notebooks, laptops, distributed systems, consoles input terminals, servers or server networks, blades, gateways, switches, processing devices, processing entities, set-top boxes, relays, routers, network access points, base stations, the like, and/or any combination of devices or entities adapted to perform the functions, operations, and/or processes described herein. Such functions, operations, and/or processes may include, for example, transmitting, receiving, operating on, processing, displaying, storing, determining, creating/generating, monitoring, evaluating, comparing, and/or similar terms used herein interchangeably. In particular embodiments, these functions, operations, and/or processes can be performed on data, content, information/data, and/or similar terms used herein interchangeably.

As indicated, in particular embodiments, the analysis computing entity 520 may also include one or more communications interfaces 528 for communicating with various computing entities, such as by communicating data, content, information/data, and/or similar terms used herein interchangeably that can be transmitted, received, operated on, processed, displayed, stored, and/or the like.

As shown in FIG. 5, in particular embodiments, the analysis computing entity 520 may include or be in communication with one or more processing elements 522 (also referred to as processors, processing circuitry, and/or similar terms used herein interchangeably) that communicate with other elements within the analysis computing entity 520 via a bus, for example. As will be understood, the processing element 522 may be embodied in a number of different ways. For example, the processing element 522 may be embodied as one or more complex programmable logic devices (CPLDs), microprocessors, multi-core processors, coprocessing entities, application-specific instruction-set processors (ASIPs), microcontrollers, and/or controllers. Further, the processing element 522 may be embodied as one or more other processing devices or circuitry. The term circuitry may refer to an entirely hardware embodiment or a combination of hardware and computer program products. Thus, the processing element 522 may be embodied as integrated circuits, application specific integrated circuits (ASICs), field programmable gate arrays (FPGAs), programmable logic arrays (PLAs), hardware accelerators, other circuitry, and/or the like. As will therefore be understood, the processing element 522 may be configured for a particular use or configured to execute instructions stored in volatile or non-volatile media or otherwise accessible to the processing element 522. As such, whether configured by hardware or computer program products, or by a combination thereof, the processing element 522 may be capable of performing steps or operations according to embodiments of the present disclosure when configured accordingly.

In particular embodiments, the analysis computing entity 520 may further include or be in communication with non-volatile media 524 (also referred to as non-volatile storage, memory, memory storage, memory circuitry and/or similar terms used herein interchangeably). In particular embodiments, the non-volatile media 524 may include one or more non-volatile storage or memory media, including but not limited to hard disks, ROM, PROM, EPROM, EEPROM, flash memory, MMCs, SD memory cards, Memory Sticks, CBRAM, PRAM, FeRAM, NVRAM, MRAM, RRAM, SONOS, FJG RAM, Millipede memory, racetrack memory, and/or the like. As will be recognized, the non-volatile media 524 may store databases (e.g., parcel/item/shipment database), database instances, database management systems, data, applications, programs, program modules, scripts, source code, object code, byte code, compiled code, interpreted code, machine code, executable instructions, and/or the like. The term database, database instance, database management system, and/or similar terms used herein interchangeably may refer to a collection of records or information/data that is stored in a computer-readable storage medium using one or more database models, such as a hierarchical database model, network model, relational model, entity-relationship model, object model, document model, semantic model, graph model, and/or the like.

In particular embodiments, the analysis computing entity 520 may further include or be in communication with volatile media 526 (also referred to as volatile storage, memory, memory storage, memory circuitry and/or similar terms used herein interchangeably). In particular embodiments, the volatile media 526 may also include one or more volatile storage or memory media, including but not limited to RAM, DRAM, SRAM, FPM DRAM, EDO DRAM, SDRAM, DDR SDRAM, DDR2 SDRAM, DDR3 SDRAM, RDRAM, TTRAM, T-RAM, Z-RAM, RIMM, DIMM, SIMM, VRAM, cache memory, register memory, and/or the like. As will be recognized, the volatile media 526 may be used to store at least portions of the databases, database instances, database management systems, data, applications, programs, program modules, scripts, source code, object code, byte code, compiled code, interpreted code, machine code, executable instructions, and/or the like being executed by, for example, the processing element 522. Thus, the databases, database instances, database management systems, data, applications, programs, program modules, scripts, source code, object code, byte code, compiled code, interpreted code, machine code, executable instructions, and/or the like may be used to control certain aspects of the operation of the analysis computing entity 520 with the assistance of the processing element 522 and operating system.

As indicated, in particular embodiments, the analysis computing entity 520 may also include one or more communications interfaces 528 for communicating with various computing entities, such as by communicating information/data, content, information/data, and/or similar terms used herein interchangeably that can be transmitted, received, operated on, processed, displayed, stored, and/or the like. Such communication may be executed using a wired information/data transmission protocol, such as fiber distributed information/data interface (FDDI), digital subscriber line (DSL), Ethernet, asynchronous transfer mode (ATM), frame relay, information/data over cable service interface specification (DOCSIS), or any other wired transmission protocol. Similarly, the analysis computing entity 520 may be configured to communicate via wireless external communication networks using any of a variety of protocols, such as general packet radio service (GPRS), Universal Mobile Telecommunications System (UMTS), Code Division Multiple Access 2000 (CDMA2000), CDMA2000 1× (1×RTT), Wideband Code Division Multiple Access (WCDMA), Time Division-Synchronous Code Division Multiple Access (TD-SCDMA), Long Term Evolution (LTE), Evolved Universal Terrestrial Radio Access Network (E-UTRAN), Evolution-Data Optimized (EVDO), High Speed Packet Access (HSPA), High-Speed Downlink Packet Access (HSDPA), IEEE 802.11 (Wi-Fi), Wi-Fi Direct, 802.16 (WiMAX), ultra wideband (UWB), infrared (IR) protocols, near field communication (NFC) protocols, Wibree, Bluetooth protocols, wireless universal serial bus (USB) protocols, long range low power (LoRa), LTE Cat M1, NarrowBand IoT (NB IoT), and/or any other wireless protocol.

Although not shown, the analysis computing entity 520 may include or be in communication with one or more input elements, such as a keyboard input, a mouse input, a touch screen/display input, motion input, movement input, audio input, pointing device input, joystick input, keypad input, and/or the like. The analysis computing entity 520 may also include or be in communication with one or more output elements (not shown), such as audio output, video output, screen/display output, motion output, movement output, and/or the like.

As will be appreciated, one or more of the analysis computing entity's 520 components may be located remotely from other analysis computing entity 520 components, such as in a distributed system. Furthermore, one or more of the components may be combined and additional components performing functions described herein may be included in the analysis computing entity 520. Thus, the analysis computing entity 520 can be adapted to accommodate a variety of needs and circumstances. As will be recognized, these architectures and descriptions are provided for exemplary purposes only and are not limiting to the various embodiments.

### Example Source Computing Entity

Turning now to FIG. 6, a block diagram of a source computing entity 530 of FIG. 5, according to some embodiments. In certain embodiments, source computing entity(s) 530 may be embodied as handheld computing entities, such as mobile phones, tablets, personal digital assistants, and/or the like, that may be operated at least in part based on user input received from a user via an input mechanism. Moreover, source computing entity(s) 530 may be embodied as onboard vehicle computing entities, such as central vehicle electronic control units (ECUs), onboard multimedia system, and/or the like that may be operated at least in part based on user input. Such onboard vehicle computing entities may be configured for autonomous and/or nearly autonomous operation however, as they may be embodied as onboard control systems for autonomous or semi-autonomous vehicles, such as unmanned aerial vehicles (UAVs), robots, and/or the like. As a specific example, source computing entity(s) 530 may be utilized as onboard controllers for UAVs configured for picking-up and/or delivering passengers to various locations, and accordingly such source computing entity(s) 530 may be configured to monitor various inputs (e.g., from various sensors) and generated various outputs. It should be understood that various embodiments of the present disclosure may comprise a plurality of source computing entities 530 embodied in one or more forms (e.g., kiosks, mobile devices, watches, laptops, tablets, smartphones, smartwatches, and the like.)

As will be recognized, a user may be an individual, a family, a company, an organization, an entity, a passenger, a department within an organization, a representative of an organization and/or person, and/or the like—whether associated with a source computing entity(s) 530. In particular embodiments, a user may operate a source computing entity 530 that may include one or more components that are functionally similar to those of the analysis computing entity 520. This figure provides an illustrative schematic representative of a source computing entity(s) 530 that can be used in conjunction with embodiments of the present disclosure. In general, the terms device, system, source computing entity, user device, entity, and/or similar words used herein interchangeably may refer to, for example, one or more computers, computing entities, desktops, mobile phones, tablets, phablets, notebooks, laptops, distributed systems, vehicle multimedia systems, autonomous vehicle onboard control systems, watches, glasses, key fobs, radio frequency identification (RFID) tags, ear pieces, scanners, imaging devices/cameras (e.g., part of a multi-view image capture system), wristbands, kiosks, input terminals, servers or server networks, blades, gateways, switches, processing devices, processing entities, set-top boxes, relays, routers, network access points, base stations, the like, and/or any combination of devices or entities adapted to perform the functions, operations, and/or processes described herein. Source computing entity(s) 530 can be operated by various parties, including personnel (passengers, network administrators, flight personnel, pilots, and/or the like). As shown in FIG. 6, the source computing entity(s) 530 can include an antenna 531, a transmitter 532 (e.g., radio), a receiver 534 (e.g., radio), and a processing element 536 (e.g., CPLDs, microprocessors, multi-core processors, coprocessing entities, ASIPs, microcontrollers, and/or controllers) that provides signals to and receives signals from the transmitter 532 and receiver 534, respectively. In some embodiments, the source computing entity(s) 530 additionally includes other components not shown, such as a fingerprint reader, a printer, and/or the camera.

The signals provided to and received from the transmitter 532 and the receiver 534, respectively, may include signaling information in accordance with air interface standards of applicable wireless systems. In this regard, the source computing entity(s) 530 may be capable of operating with one or more air interface standards, communication protocols, modulation types, and access types. More particularly, the source computing entity(s) 530 may operate in accordance with any of a number of wireless communication standards and protocols, such as those described above with regard to the analysis computing entity(s) 520. In a particular embodiment, the source computing entity(s) 530 may operate in accordance with multiple wireless communication standards and protocols, such as UMTS, CDMA2000, 1×RTT, WCDMA, TD-SCDMA, LTE, E-UTRAN, EVDO, HSPA, HSDPA, Wi-Fi, Wi-Fi Direct, WiMAX, UWB, IR, NFC, Bluetooth, USB, and/or the like. Similarly, the source computing entity(s) 530 may operate in accordance with multiple wired communication standards and protocols, such as those described above with regard to the analysis computing entity(s) 520 via a network interface 444.

Via these communication standards and protocols, the source computing entity(s) 530 can communicate with various other entities using concepts such as Unstructured Supplementary Service information/data (USSD), Short Message Service (SMS), Multimedia Messaging Service (MMS), Dual-Tone Multi-Frequency Signaling (DTMF), and/or Subscriber Identity Module Dialer (SIM dialer). The source computing entity(s) 530 can also download changes, add-ons, and updates, for instance, to its firmware, software (e.g., including executable instructions, applications, program modules), and operating system.

According to particular embodiments, the source computing entity(s) 530 may include location determining aspects, devices, modules, functionalities, and/or similar words used herein interchangeably. For example, the source computing entity(s) 530 may include outdoor positioning aspects, such as a location module adapted to acquire, for example, latitude, longitude, altitude, geocode, course, direction, heading, speed, universal time (UTC), date, and/or various other information/data. In particular embodiments, the location module can acquire information/data, sometimes known as ephemeris information/data, by identifying the number of satellites in view and the relative positions of those satellites (e.g., using global positioning systems (GPS)). The satellites may be a variety of different satellites, including Low Earth Orbit (LEO) satellite systems, Department of Defense (DOD) satellite systems, the European Union Galileo positioning systems, the Chinese Compass navigation systems, Indian Regional Navigational satellite systems, and/or the like. This information/data can be collected using a variety of coordinate systems, such as the DecimalDegrees (DD); Degrees, Minutes, Seconds (DMS); Universal Transverse Mercator (UTM); Universal Polar Stereographic (UPS) coordinate systems; and/or the like. Alternatively, the location information can be determined by triangulating the computing entity's 530 position in connection with a variety of other systems, including cellular towers, Wi-Fi access points, and/or the like. Similarly, the source computing entity(s) 530 may include indoor positioning aspects, such as a location module adapted to acquire, for example, latitude, longitude, altitude, geocode, course, direction, heading, speed, time, date, and/or various other information/data. Some of the indoor systems may use various position or location technologies including RFID tags, indoor beacons or transmitters, Wi-Fi access points, cellular towers, nearby computing devices/entities (e.g., smartphones, laptops) and/or the like. For instance, such technologies may include the iBeacons, Gimbal proximity beacons, Bluetooth Low Energy (BLE) transmitters, NFC transmitters, and/or the like. These indoor positioning aspects can be used in a variety of settings to determine the location of someone or something to within inches or centimeters.

The source computing entity(s) 530 may also comprise a user interface (that can include a display 538 coupled to a processing element 536) and/or a user input interface (coupled to a processing element 536). For example, the user interface may be a user application, browser, user interface, and/or similar words used herein interchangeably executing on and/or accessible via the source computing entity 530 to interact with and/or cause display of information from the analysis computing entity 520, as described herein. The user input interface can comprise any of a number of devices or interfaces allowing the source computing entity(s) 530 to receive information/data, such as a keypad 540 (hard or soft), a touch display, voice/speech or motion interfaces, or other input device. In embodiments, the keypad 540 can include (or cause display of) the conventional numeric (0-9) and related keys (#, *), and other keys used for operating the source computing entity(s) 530 and may include a full set of alphabetic keys or set of keys that may be activated to provide a full set of alphanumeric keys. In addition to providing input, the user input interface can be used, for example, to activate or deactivate certain functions, such as screen savers and/or sleep modes.

As shown in this figure, the source computing entity(s) 530 may also include a camera 542, imaging device, and/or similar words used herein interchangeably (e.g., still-image camera, video camera, IoT enabled camera, IoT module with a low resolution camera, a wireless enabled MCU, and/or the like) configured to capture images. The source computing entity(s) 530 may be configured to capture images via the onboard camera 542, and to store those imaging devices/cameras locally, such as in the volatile memory 546 and/or non-volatile memory 548. As discussed herein, the source computing entity(s) 530 may be further configured to match the captured image data with relevant location and/or time information captured via the location determining aspects to provide contextual information/data, such as a time-stamp, date-stamp, location-stamp, and/or the like to the image data reflective of the time, date, and/or location at which the image data was captured via the camera 542. The contextual data may be stored as a portion of the image (such that a visual representation of the image data includes the contextual data) and/or may be stored as metadata associated with the image data that may be accessible to various computing entity(s) 530.

The source computing entity(s) 530 may include other input mechanisms, such as sensor devices (e.g., physiological sensor devices), microphones, accelerometers, RFID readers, and/or the like configured to capture and store various information types for the source computing entity(s) 530. For example, the sensor devices can include sensors such as an accelerometer, gyroscope, magnetometer or digital compass, a proximity sensor, an ambient light sensor, a barometer, a temperature sensor, a humidity sensor, a fingerprint sensor, a heart rate sensor, a GPS sensor, and the like.

The source computing entity(s) 530 can also include volatile memory 546 and/or non-volatile storage or memory 548, which can be embedded and/or may be removable. For example, the non-volatile memory may be ROM, PROM, EPROM, EEPROM, flash memory, MMCs, SD memory cards, Memory Sticks, CBRAM, PRAM, FeRAM, NVRAM, MRAM, RRAM, SONOS, FJG RAM, Millipede memory, racetrack memory, and/or the like. The volatile memory may be RAM, DRAM, SRAM, FPM DRAM, EDO DRAM, SDRAM, DDR SDRAM, DDR2 SDRAM, DDR3 SDRAM, RDRAM, TTRAM, T-RAM, Z-RAM, RIMM, DIMM, SIMM, VRAM, cache memory, register memory, and/or the like. The volatile and non-volatile storage or memory can store databases, database instances, database management systems, information/data, applications, programs, program modules, scripts, source code, object code, byte code, compiled code, interpreted code, machine code, executable instructions, and/or the like to implement the functions of the source computing entity(s) 530. As indicated, this may include a user application that is resident on the entity or accessible through a browser or other user interface for communicating with the analysis computing entity 520 and/or various other computing entities.

In another embodiment, the source computing entity(s) 530 may include one or more components or functionality that are the same or similar to those of the analysis computing entity 520, as described in greater detail above. As will be recognized, these architectures and descriptions are provided for exemplary purposes only and are not limiting to the various embodiments.

As used herein, the term “determine” or “determining” encompasses a wide variety of actions and, therefore, “determining” can include calculating, computing, processing, deriving, investigating, looking up (such as via looking up in a table, a database or another data structure), inferring, ascertaining, measuring, and the like. Also, “determining” can include receiving (such as receiving information), accessing (such as accessing data stored in memory), transmitting (such as transmitting information), and the like. Also, “determining” can include resolving, selecting, obtaining, choosing, establishing, and other similar actions.

As used herein, a phrase referring to “at least one of” a list of items refers to any combination of those items, including single members. As an example, “at least one of: a, b, or c” is intended to cover: a, b, c, a-b, a-c, b-c, and a-b-c. As used herein, “or” is intended to be interpreted in the inclusive sense, unless otherwise explicitly indicated. For example, “a or b” may include a only, b only, or a combination of a and b.

As used herein, “based on” is intended to be interpreted in the inclusive sense, unless otherwise explicitly indicated. For example, “based on” may be used interchangeably with “based at least in part on,” “associated with”, or “in accordance with” unless otherwise explicitly indicated. Specifically, unless a phrase refers to “based on only ‘a,’” or the equivalent in context, whatever it is that is “based on ‘a,’” or “based at least in part on ‘a,’” may be based on “a” alone or based on a combination of “a” and one or more other factors, conditions or information.

The various illustrative components, logic, logical blocks, modules, circuits, operations, and algorithm processes described in connection with the examples disclosed herein may be implemented as electronic hardware, firmware, software, or combinations of hardware, firmware, or software, including the structures disclosed in this specification and the structural equivalents thereof. The interchangeability of hardware, firmware, and software has been described generally in terms of functionality and illustrated in the various illustrative components, blocks, modules, circuits, and processes described above. Whether such functionality is implemented in hardware, firmware, or software depends upon the particular application and design constraints imposed on the overall system.

Various modifications to the examples described in this disclosure may be readily apparent to persons having ordinary skill in the art, and the generic principles defined herein may be applied to other examples without departing from the spirit or scope of this disclosure. Thus, the claims are not intended to be limited to the examples shown herein but are to be accorded the widest scope consistent with this disclosure, the principles, and the novel features disclosed herein.

Additionally, various features that are described in this specification in the context of separate examples also can be implemented in combination in a single implementation. Conversely, various features that are described in the context of a single implementation can also be implemented in multiple examples separately or in any suitable subcombination. As such, although features may be described above as acting in particular combinations, and even initially claimed as such, one or more features from a claimed combination can, in some cases, be excised from the combination, and the claimed combination may be directed to a subcombination or variation of a subcombination.

Similarly, while operations are depicted in the drawings in a particular order, this should not be understood as requiring that such operations be performed in the particular order shown or in sequential order, or that all illustrated operations be performed, to achieve desirable results. Further, the drawings may schematically depict one or more example processes in the form of a flowchart or flow diagram. However, other operations that are not depicted can be incorporated into the example processes that are schematically illustrated. For example, one or more additional operations can be performed before, after, simultaneously, or between any of the illustrated operations. In some circumstances, multitasking and parallel processing may be advantageous. Moreover, the separation of various system components in the examples described above should not be understood as requiring such separation in all examples, and it should be understood that the described program components and systems can generally be integrated together in a single software product or packaged into multiple software products.

What is claimed is:

1. A method of mitigating aircraft passenger anxiety during travel, the method comprising:
receiving sensor data associated with physiological measurements of a passenger within an aircraft;
determining a state of the passenger based, at least in part, on the physiological measurements of the passenger;
calculating a remedial action associated with the state of the passenger and the aircraft; and
implementing the remedial action on the aircraft to alter the state of the passenger.

2. The method of claim 1, wherein the aircraft is an electric Vertical Takeoff and Landing (eVTOL) aircraft.

3. The method of claim 1, wherein the remedial action includes altering a flight envelope of the aircraft.

4. The method of claim 1, wherein the remedial action includes rerouting a flight path of the aircraft.

5. The method of claim 1, wherein calculating the remedial action includes:
inputting the sensor data and information associated with the aircraft and a travel route into a machine learning model trained on historical data associated with the aircraft and the passenger;
producing, by the machine learning model, a remedial action inference; and
utilizing the remedial action inference as the remedial action.

6. The method of claim 5, wherein the historical data includes information relating to previous passenger stress indicators along the travel route.

7. The method of claim 1, wherein current weather conditions of a travel route of the aircraft is factored when calculating the remedial action.

8. The method of claim 1, wherein the sensor data is received from a plurality of sensors positioned in and around the aircraft.

9. The method of claim 1, wherein the physiological measurements includes a blood pressure, a heart rate, a blood oxygen level, and a body temperature associated with the passenger.

10. The method of claim 1, wherein the sensor data is received from a personal device associated with the passenger.

11. The method of claim 10, wherein the personal device is paired to the aircraft and configured to transmit the sensor data to the aircraft.

12. The method of claim 1, further comprising:
receiving additional sensor data associated with the physiological measurements of the passenger after the remedial action is implemented on the aircraft;
determining an updated state of the passenger, based, at least in part, on the physiological measurements of the passenger received from the additional sensor data;
calculating an updated remedial action associated with the updated state of the passenger and the remedial action previously implemented; and
implementing the updated remedial action on the aircraft to alter the updated state of the passenger.

13. A vehicle comprising:
one or more memories that store processor-executable code; and
one or more processors coupled with the one or more memories and individually or collectively configured to, in association with executing the code, cause the vehicle to:
determine a state of a passenger based, at least in part, on sensor data associated with physiological measurements of the passenger in the vehicle;
calculate a remedial action associated with the state of the passenger and the vehicle; and
implement the remedial action to the vehicle to alter the state of the passenger.

14. The vehicle of claim 13, wherein the vehicle is an aircraft.

15. The vehicle of claim 14, wherein the aircraft is an electric Vertical Takeoff and Landing (eVTOL) aircraft.

16. The vehicle of claim 13, wherein the remedial action includes altering a flight path of the vehicle.

17. The vehicle of claim 13, wherein the remedial action includes rerouting a flight path of the vehicle.

18. The vehicle of claim 13, wherein calculating the remedial action cause the vehicle to:
input the sensor data and information associated with the vehicle and a travel route into a machine learning model trained on historical data associated with the vehicle and the passenger;
produce, by the machine learning model, a remedial action inference; and
utilize the remedial action inference as the remedial action.

19. One or more computer storage media storing computer-useable instructions that, when executed by one or more computing devices, cause the one or more computing devices to perform operations comprising:
receiving information associated with an aircraft and a passenger within the aircraft;
predicting a source of passenger stress associated with the information and based, at least in part, to the passenger and the aircraft;
calculating a preemptive remedial action associated with the source of passenger stress; and
implementing the preemptive remedial action on the aircraft.

20. The one or more computer storage media of claim 19, wherein predicting the source of passenger stress includes:
inputting the information associated with the aircraft into a machine learning model trained on historical data associated with the aircraft and the passenger;
producing, by the machine learning model, a source of stress inference; and
utilizing the source of stress inference as the source of passenger stress.
